(19) United States
(12) Patent Application Publication
(10) Pub. No.: US 2019/0062452 A1
(43) Pub. Date: Feb. 28, 2019

(54) Methods and Compositions for the Treatment of Immunomodulatory Diseases and Disorders

(71) Applicant: LANKENAU INSTITUTE FOR MEDICAL RESEARCH, Wynnewood, PA (US)

(72) Inventors: Lisa Laury-Kleintop, Ambler, PA (US); Laura Mandik-Nayak, Plymouth Meeting, PA (US); Lauren M.F. Merlo, Wynnewood, PA (US); James B. DuHadaway, Wilmington, DE (US); George C. Prendergast, Penn Valley, PA (US)

(21) Appl. No.: 15/742,972

(22) PCT Filed: Jul. 27, 2016
(86) PCT No.: PCT/US2016/044230

§ 371 Date: Jan. 9, 2018

Related U.S. Application Data
(60) Provisional application No. 62/197,900, filed on Jul. 28, 2015.

(51) Int. Cl. C07K 16/40; C12N 15/115; A61K 39/395; A61K 45/06; A61P 19/02

(52) U.S. Cl. CPC C07K 16/40; C12N 15/115; A61K 39/3955; A61K 2039/505; A61P 19/02; C12N 2310/16; A61K 45/06

(57) ABSTRACT

Compositions and methods for the treatment of an autoantibody disease or disorder are disclosed.

This application claims priority under 35 U.S.C. § 119(e) to U.S. Provisional Patent Application No. 62/197,900, filed on Jul. 28, 2015. The foregoing application is incorporated by reference herein.

### FIELD OF THE INVENTION

This invention relates generally to the field of immunotherapy. Specifically, the invention provides novel compositions and methods for the treatment of diseases and disorders by administration of anti-IDO2 molecules.

### BACKGROUND OF THE INVENTION

Pathogenic drivers of autoimmunity remain a major focus of research aiming to reduce morbidity and mortality in patients who suffer from autoimmune disease. Therapeutic strategies to relieve or reprogram inflammation and deplete autoantibodies or B cell populations have been explored with variable clinical success (Townsend et al., Immunol. Rev. (2010) 237: 264-283; Harvey et al., BioDrugs (2013) 27:85-95; Buch et al., Ann. Rheum. Dis. (2011) 70:909-920). However, new strategies that target the underlying mechanisms driving autoimmune responses are still urgently needed.

### SUMMARY OF THE INVENTION

In accordance with one aspect of the instant invention, methods for inhibiting, treating, and/or preventing autoantibody related diseases or disorders (e.g., an autoimmune disease) in a subject in need thereof are provided. The invention also encompasses methods for reducing autoantibody production in a subject. The methods comprise the administration of at least one aptamer and/or antibody or antibody fragment immunologically specific for IDO2. In a particular embodiment, the methods comprise the administration of a composition comprising at least one aptamer and/or antibody or antibody fragment immunologically specific for IDO2 and at least one pharmaceutically acceptable carrier. In a particular embodiment, the methods further comprise the administration of at least one other therapeutic agent or method for treating, inhibiting, or preventing the autoimmune disease concurrently and/or sequentially with at least one antibody or antibody fragment immunologically specific for IDO2.

Compositions comprising at least one anti-IDO2 antibody (or fragment thereof) and/or anti-IDO2 aptamer and at least one pharmaceutically acceptable carrier are also provided. The composition may further comprise at least one other therapeutic agent.

### BRIEF DESCRIPTION OF THE DRAWINGS

FIG. 1 provides a graph of the number of autoantibody secreting cells in mice treated with control mouse antibodies or anti-IDO2 antibodies. Graph shows mean ±SEM for n=13 control Ig and n=11 anti-IDO2 Ig treated mice.

FIG. 2 provides a graph of mean ankle thickness in mice treated with control mouse antibodies or anti-IDO2 antibodies. Graph shows mean ±SEM for n=13 control Ig and n=11 anti-IDO2 Ig treated mice.

FIG. 3 provides a graph of the number of autoantibody secreting cells in wild-type (KRN.g7) or IDO2 knockout KRN.g7 (IDO2 ko) mice treated with control mouse antibodies or anti-IDO2 antibodies. Graph shows mean ±SEM for n=6 KRN.g7 and n=8 IDO2 knockout KRN.g7 mice per treatment.

FIG. 4 provides a graph of mean ankle thickness in wild-type (KRN.g7) or IDO2 knockout KRN.g7 (IDO2 ko) mice treated with control mouse antibodies or anti-IDO2 antibodies. Graph shows mean ±SEM for n=6 KRN.g7 and n=8 IDO2 knockout KRN.g7 mice per treatment.

FIG. 5 provides a graph of the number of autoantibody secreting cells in wild-type (KRN.g7) or IDO2 knockout KRN.g7 (IDO2 ko) mice treated with control mouse antibodies or anti-IDO2 antibodies at 21 (pre-arthritis) or 28 (post-arthritis) days of age. At 6 weeks of age, the joint draining lymph nodes were harvested and analyzed for the number of autoantibody secreting cells (ASCs) by Enzyme-linked immunospot (ELISpot) assay. Graph shows mean ±SEM for n=15 control Ig, n=11 pre-arthritis, and n=9 post-arthritis anti-IDO2 Ig treated mice.

FIG. 6 provides a graph of mean ankle thickness in wild-type (KRN.g7) or IDO2 knockout KRN.g7 (IDO2 ko) mice treated with control mouse antibodies or anti-IDO2 antibodies at 21 (pre-arthritis) or 28 (post-arthritis) days of age and followed for the development of arthritis. Graph shows mean ±SEM for n=9 control Ig, n=5 pre-arthritis, and n=9 post-arthritis anti-IDO2 Ig treated mice.

### DETAILED DESCRIPTION OF THE INVENTION

Herein, it is demonstrated that the enzyme indoleamine 2,3-dioxygenase 2 (IDO2) is responsible for driving autoantibody production. In particular, it is demonstrated that IDO2 drives autoantibody production in a preclinical model of rheumatoid arthritis (RA). Administration of anti-IDO2 antibodies to the mouse model of RA blocked IDO2 function and alleviated the disease. Indeed, anti-IDO2 antibody treatment was effective at reducing autoantibody levels and delaying the onset and attenuating the overall severity of arthritis compared to mice treated with control antibody. The specificity of the antibody was confirmed by the lack of response in mice genetically deficient for IDO2.

The anti-IDO2 antibodies can be used to reduce autoantibody production and/or treat any disease or disorder that is caused by or exacerbated by the accumulation of an antibody (or antibodies) directed against a patient's own tissues or cells (i.e., autoantibodies)—a common characteristic of autoimmune disease. Such diseases are mediated by the development of autoreactive immune B cells that secrete antibody. While approaches to eradicate B cells or blunt their action have been developed, the use of the anti-IDO2 antibody approach of the instant invention provides a unique manner to limit antibody secretion by the autoreactive B cell. By blunting production of autoimmune antibodies, the methods of the instant invention do not displace disease-specific approaches that may be developed and utilized. For example, therapeutics that alleviate the inflammatory reaction induced by the autoimmune response may be co-administered with the anti-IDO2 antibody therapy to provide a cooperative or even synergistic effect against the disease. Thus, the anti-IDO2 antibody therapeutic methods offer significant advantages in terms of its simplicity and general utility, but also in its unique mechanism and ability to be combined with other therapeutic approaches.

Another beneficial feature of the instant invention is its expected low toxicity or side-effects, as observed in the in vivo experiments presented herein. Notably, mice that are genetically deficient for IDO2 are normal and lack evident immune deficiencies, including deficiencies in B cell responses to antigen stimulation or IgG memory formation, which has been explicitly tested. Thus, the anti-IDO2 antibody technology retards abnormal B cell function in the production of autoimmune antibodies, but has not been shown to disrupt normal B cell function after canonical antigenic challenge.

In certain cases, the application of the anti-IDO2 antibody may be similar to other antibody-based therapies which are tolerable despite their non-targeted aspect for disease treatment. Examples include, but are not limited to, the antibody therapies anti-TNFα (infliximab, adalimumab, etanercept), anti-CD20 (rituximab), and anti-BLyS (belimumab), all of which generally blunt inflammation or eradicate B cells or B cell function. For patients that poorly tolerate these therapies, the anti-IDO2 antibody provides another therapeutic option.

The present invention provides compositions and methods for the inhibition, prevention, and/or treatment of an autoimmune disease. The methods comprise administering at least one anti-IDO2 antibody (and/or anti-IDO2 aptamer (see below)) to a subject. The methods of the instant invention may further comprise the administration of at least one other therapeutic for the disease being treated. For example, the anti-IDO2 antibodies or aptamers may be co-administered with an anti-inflammatory agent and/or immunosuppressant. The agents administered to the subject may be contained within a composition comprising at least one pharmaceutically acceptable carrier. When more than one agent is being administered (e.g., anti-IDO2 antibody or aptamer with an additional therapeutic), the agents may be administered consecutively (before or after) and/or at the same time (concurrently). The agents may be administered in the same composition or in separate compositions.

As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system responds to self antigens and mediates cell and tissue damage. In a particular embodiment, autoimmune diseases are characterized as being a result of, at least in part, a humoral immune response. In a particular embodiment, the autoimmune disease is T-cell dependent (e.g., T cell help or crosstalk to B cells for autoantibody production). Examples of autoimmune disease include, without limitation: acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyradiculoneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devie's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalopathy, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paraneoplastic neurological syndrome, paroxysmal nocturnal hemoglobinuria (PNH), Parry Romberg syndrome, Parsonnage-Turner syndrome, pars planitis (peripheral uveitis), pemphigus (pemphigus vulgaris), peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)). In a particular embodiment, the autoimmune disease is selected from the group consisting of rheumatoid arthritis, Type 1 diabetes, systemic lupus erythematosus, myasthenia gravis, multiple sclerosis, scleroderma, Addison's disease, bullous pemphigoid, pemphigus vulgaris, Guillain-Barré syndrome, Sjogren syndrome, dermatomyositis, thrombotic thrombocytopenic purpura, monoclonal gammopathy of undetermined significance, Waldenstrom's macroglobulinemia, chronic inflammatory demyelinating polyradiculoneuropathy, Hashimoto's encephalopathy, Hashimoto's thyroiditis, Graves' disease, Wegener's granulomatosis, and antibody-mediated transplantation rejection. In a particular embodiment, the autoimmune disease is rheumatoid arthritis. When the autoimmune disease is rheumatoid arthritis, the methods of the instant invention may further comprise the administration of at least one other rheumatoid arthritis therapy (e.g., an anti-inflammatory (e.g., methotrexate), B cell depletion therapy, and/or small molecule inhibitors of the IDO pathway (e.g., 1-methyl-tryptophan).

The present invention provides compositions and methods for the inhibition, prevention, and/or treatment of cancers sustained by antibody secretion (e.g., blood tumors (e.g., multiple myeloma) or solid tumors (e.g., wherein antibody secretion contributes to supportive inflammatory processes) (e.g., squamous cell carcinoma (SCC) (Affara et al., Cancer Cell (2014) 25(6):809-821). The methods comprise administering at least one anti-IDO2 antibody to a subject. The methods of the instant invention may further comprise the administration of at least one other therapeutic for the cancer being treated. For example, the methods may further comprise the administration of at least one chemotherapeutic agent and/or anti-cancer therapy (e.g., radiation therapy and/or surgery to remove cancerous cells or a tumor (e.g., resection)). The agents administered to the subject may be contained within a composition comprising at least one pharmaceutically acceptable carrier. When more than one agent is being administered (e.g., anti-IDO2 antibody with an additional chemotherapeutic), the agents may be administered consecutively (before or after) and/or at the same time (concurrently). The agents may be administered in the same composition or in separate compositions.

The present invention provides compositions and methods for the inhibition, prevention, and/or treatment of antibody-mediated paraneoplastic syndrome. Paraneoplastic syndromes are disorders associated with cancer, not caused by direct invasion, metastasis or consequences of treatment. The methods comprise administering at least one anti-IDO2 antibody to a subject. Examples of antibody-mediated paraneoplastic syndrome include, without limitation, stiff person syndrome (e.g., in breast cancer), dermatomyositis (e.g., in breast cancer), opsoclonus-myoclonus (e.g., in breast cancer), peripheral encephalomyelitis (e.g., in lung cancer), and retinopathy (e.g., in lung cancer). The methods of the instant invention may further comprise the administration of at least one other therapeutic for the paraneoplastic syndrome or cancer being treated. For example, the methods may further comprise the administration of at least one other therapeutic agent. The agents administered to the subject may be contained within a composition comprising at least one pharmaceutically acceptable carrier. When more than one agent is being administered (e.g., anti-IDO2 antibody with an additional therapeutic), the agents may be administered consecutively (before or after) and/or at the same time (concurrently). The agents may be administered in the same composition or in separate compositions.

The present invention provides compositions and methods for the inhibition, prevention, and/or treatment of antibody-mediated inflammatory diseases. The methods comprise administering at least one anti-IDO2 antibody to a subject. Examples of antibody-mediated inflammatory disease include, without limitation, allergic responses, Celiac disease, Crohn's disease, inflammatory bowel disease, rheumatoid arthristis, systemic lupus erythematosus, myasthenia gravis, scleroderma, type I diabetes, monoclonal gammopathy of undetermined significance (MGUS), Sjogren's syndrome, Waltdenstrom's macroglobulinemia and Hashimoto's thyroiditis. The methods of the instant invention may further comprise the administration of at least one other therapeutic for the inflammatory disease being treated. For example, the methods may further comprise the administration of at least one other anti-inflammatory agent. The agents administered to the subject may be contained within a composition comprising at least one pharmaceutically acceptable carrier. When more than one agent is being administered (e.g., anti-IDO2 antibody with an additional therapeutic), the agents may be administered consecutively (before or after) and/or at the same time (concurrently). The agents may be administered in the same composition or in separate compositions.

As stated hereinabove, the methods (and compositions) of the instant invention comprise administering at least one antibody or antibody fragment which is immunologically specific for IDO2 (indoleamine 2,3-dioxygenase 2; anti-IDO2 antibody) to a subject. In a particular embodiment, the anti-IDO2 antibody is immunologically specific for human IDO2. In a particular embodiment, the anti-IDO2 antibody is immunologically specific for human IDO2 to the exclusion of IDO1. Amino acid and nucleotide sequences of human IDO2 and its isoforms/variants are provided in GenBank Gene ID: 169355 and GenBank Accession Nos. NM_194294.2 and NP_919270.2. An exemplary amino acid sequence of human IDO2 (e.g., 420 amino acids) is:

| (SEQ ID NO: 1) | |
|---|---|
| 1 | MLHFHYYDTS NKIMEPHRPN VKTAVPLSLE SYHISEEYGF LLPDSLKELP |
| 51 | DHYRPWMEIA NKLPQLIDAH QLQAHVDKMP LLSCQFLKGH REQRLAHLVL |
| 101 | SFLTMGYVWQ EGEAQPAEVL PRNLALPFVE VSRNLGLPPI LVHSDLVLTN |
| 151 | WTKKDPDGFL EIGNLETIIS FPGGESLHGF ILVTALVEKE AVPGIKALVQ |
| 201 | ATNAILQPNQ EALLQALQRL RLSIQDITKT LGQMHDYVDP DIFYAGIRIF |
| 251 | LSGWKDNPAM PAGLMYEGVS QEPLKYSGGS AAQSTVLHAF DEFLGIRHSK |
| 301 | ESGDFLYRMR DYMPPSHKAF IEDIHSAPSL RDYILSSGQD HLLTAYNQCV |
| 351 | QALAELRSYH TTMVTKYLIT AAAKAKHGKP NHLPGPPQAL KDRGTGGTAV |
| 401 | MSFLKSVRDK TLESILHPRG. |

In a particular embodiment, the amino acid sequence of IDO2 has at least 80%, 85%, 90%, 95%, 97%, 99%, or 100% homology or identity with SEQ ID NO: 1.

In a particular embodiment, the anti-IDO2 antibody recognizes or is immunologically specific the mouse IDO2 epitope RDYILASGPGDCLMAYNQCVE (SEQ ID NO: 2). The anti-IDO2 antibody used in the Example recognizes this epitope. In a particular embodiment, the amino acid sequence of the light chain (leader, variable, J) is:

| (SEQ ID NO: 3) | |
|---|---|
| 1 | MSVLTQVLAL LLLWLTGARC DIQMTQSPAS LSASVGETVT ITCRASENIH |
| 51 | NYLAWYQQKQ GKSPQLLVYN PKNLADGVPS RFSGSGSGTQ YSLNINSLQP |
| 101 | EDFGTYYCQH FWNTPPTFGG GTRLEIKR. |

In a particular embodiment, the amino acid sequence of the heavy chain (leader, variable, D, J) is:

| (SEQ ID NO: 4) | |
|---|---|
| 1 | MSSPQTLNTL TLTMGWSWIF LFLLSGTAGV LSEVQLQQSG PELVKPGASV |
| 51 | QISCKTSGYT FTEYTMHWVK QSHGKSLEWL GIIHPDNGIT RYNQKFKAKA |
| 101 | TLTEDKSSRT AYMELRSLTS EDSAVYYCAR RYYGNFDYAL DYWGQGTSVT |
| 151 | VSS. |

The antibody and antibody fragment of the instant invention may comprise at least one domain from the above anti-IDO2 monoclonal antibody. For example, the antibody or antibody fragment may comprise at least one, two, three, four, five, or all six CDR domains the above anti-IDO2 monoclonal antibody. In a particular embodiment, the antibody or antibody fragment comprises at least one or both of the CDR3 domains. In a particular embodiment, the domains of the antibody or antibody fragment have at least 90%, 95%, 97%, 99%, or 100% homology or identity with the domains present in the anti-IDO2 monoclonal antibody or with SEQ ID NOs 3 and/or 4.

In a particular embodiment, the anti-IDO2 antibody recognizes or is immunologically specific for amino acids 331-351 of human IDO2

(RDYILSSGQDHLLTAYNQCVQ; SEQ ID NO: 5).

The antibodies of the instant invention may be naturally occurring or synthetic or modified (e.g., a recombinantly generated antibody; a chimeric antibody; a bispecific antibody; a humanized antibody; a camelid antibody; and the like). The antibody may comprise at least one purification tag. In a particular embodiment, the antibody is an antibody fragment. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody. The antibody may also be a protein (e.g., a fusion protein) comprising at least one antibody or antibody fragment. In a particular embodiment of the instant invention, the antibody comprises an Fc region. In a particular embodiment of the instant invention, the antibody is a monoclonal antibody.

Notably, IDO2 is an intracellular protein rather than a cell surface protein, ligand or receptor. Therefore, the anti-IDO2 antibody may be entering cells through the Fc receptor. This mechanism of entry will result in reduced toxicity or side-effects since only cells expressing the Fc receptor will be susceptible to anti-IDO2 antibodies. Accordingly, in a particular embodiment of the instant invention, the anti-IDO2 antibody fragment comprises an Fc region. In a particular embodiment, the antibody or fragment thereof is conjugated or linked to a cell penetrating peptide, particularly when the antibody fragment does not contain an Fc region.

The instant invention also encompasses synthetic proteins which mimic an immunoglobulin. Examples include, without limitation, Affibody® molecules (Affibody, Bromma, Sweden), darpins (designed ankyrin repeat proteins; Kawe et al. (2006) J. Biol. Chem., 281:40252-40263), and peptabodies (Terskikh et al. (1997) PNAS 94:1663-1668).

The antibodies of the instant invention may be further modified. For example, the antibodies may be humanized. In a particular embodiment, the hybrid antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one detectable agent, imaging agent, contrast agent, or therapeutic compound (e.g., see above). The antibodies of the instant invention may also comprise at least one purification tag (e.g., a His-tag).

The antibody molecules of the invention may be prepared using a variety of methods known in the art. Polyclonal and monoclonal antibodies may be prepared as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

Compositions comprising at least one anti-IDO2 antibody are also encompassed by the instant invention. In a particular embodiment, the composition comprises at least one anti-IDO2 antibody or antibody fragment and at least one pharmaceutically acceptable carrier. The composition may further comprise at least one other therapeutic compound for the inhibition, treatment, and/or prevention of the disease or disorder to be treated (see, e.g., hereinabove). Alternatively, at least one other therapeutic compound may be contained within a separate composition(s) with at least one pharmaceutically acceptable carrier. The present invention also encompasses kits comprising a first composition comprising at least one anti-IDO2 antibody or antibody fragment and a second composition comprising at least one other therapeutic compound for the inhibition, treatment, and/or prevention of the disease or disorder to be treated. The first and second compositions may further comprise at least one pharmaceutically acceptable carrier.

As explained hereinabove, the compositions of the instant invention are useful for treating autoantibody related diseases or disorders. A therapeutically effective amount of the composition may be administered to the subject. The dosages, methods, and times of administration are readily determinable by persons skilled in the art, given the teachings provided herein.

The antibodies as described herein will generally be administered to a patient as a pharmaceutical preparation. The term “patient” as used herein refers to human or animal subjects. These antibodies may be employed therapeutically, under the guidance of a physician for the treatment of the indicated disease or disorder.

The pharmaceutical preparation comprising the antibody molecules of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The dose and dosage regimen of an antibody according to the invention that is suitable for administration to a particular patient may be determined by a physician considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

Selection of a suitable pharmaceutical preparation depends upon the method of administration chosen. For example, the antibodies of the invention may be administered by direct injection into any desired tissue or into the surrounding area. In this instance, a pharmaceutical preparation comprises the antibody molecules dispersed in a medium that is compatible with the target tissue.

Antibodies may also be administered parenterally, by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the antibodies, or the pharmaceutical preparation in which they are delivered, may have to be increased so that the molecules can arrive at their target locations. Furthermore, the antibodies may have to be delivered in a cell-targeting carrier so that sufficient numbers of molecules will reach the target cells. Methods for increasing the lipophilicity of a molecule are known in the art. If a small form of the antibody is to be administered, including but not limited to a Fab fragment, a Dab, an scFv or a diabody, it may be conjugated to a second (carrier) molecule such as, but not limited to polyethylene glycol (PEG) or an albumin-binding antibody or peptide to prolong its retention in blood.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of anti-IDO2 antibody molecules may be determined by evaluating the toxicity of the antibody molecules in animal models. Various concentrations of antibody pharmaceutical preparations may be administered to murine models of the disease or disorder and the minimal and maximal dosages may be determined based on the results and side effects as a result of the treatment. Appropriate dosage unit may also be determined by assessing the efficacy of the antibody molecule treatment in combination with other standard drugs. The dosage units of anti-IDO2 antibody molecules may be determined individually or in combination with another treatment.

The pharmaceutical preparation comprising the anti-IDO2 antibody molecules may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The methods of the instant invention may further comprise monitoring the disease or disorder in the subject after administration of the composition(s) of the instant invention to monitor the efficacy of the method.

While the application generally describes the use of anti-IDO2 antibodies hereinabove, the instant invention also encompasses the use of anti-IDO2 aptamers, particularly anti-IDO2 nucleic acid aptamers. In other words, the anti-IDO2 antibodies in the methods and compositions of the instant invention can be replaced or combined with an anti-IDO2 aptamer. In a particular embodiment, the anti-IDO2 aptamer recognizes or specifically binds amino acids 331-3M of human IDO2

(RDYILSSGQDHLLTAYNQCVQ; SEQ ID NO: 5).

As used herein, the term “aptamer” refers to a molecule (e.g., an oligonucleotide or peptide) that binds to a specific target molecule. In a particular embodiment, the aptamer is a nucleic acid that specifically binds to a target, such as a protein, through interactions other than Watson-Crick base pairing. In a particular embodiment, the aptamer specifically binds to one or more targets (e.g., a protein or protein complex) to the general exclusion of other molecules in a sample. The aptamer may be a nucleic acid such as an RNA, a DNA, a modified nucleic acid, or a mixture thereof. The aptamer may also be a nucleic acid in a linear or circular form and may be single stranded or double stranded. The aptamer may comprise oligonucleotides that are at least 5, at least 10, at least 15, at least 20, at least 25, at least 30, at least 35, at least 40 or more nucleotides in length. Aptamers may comprise sequences that are up to 40, up to 60, up to 80, up to 100, up to 150, up to 200 or more nucleotides in length. Aptamers may be from about 5 to about 150 nucleotides, from about 10 to about 100 nucleotides, or from about 20 to about 75 nucleotides in length. While aptamers are generally nucleic acid molecules (e.g., oligonucleotides), aptamer equivalents may also be used in place of the nucleic acid aptamers, such as peptide aptamers. Nucleic acid aptamers can be naturally occurring. However, most nucleic acid aptamers are derived from diverse combinatorial libraries using Systematic Evolution of Ligands by Exponential enrichment (SELEX) as well as a cell-based version called Cell SELEX (Sundaram et al. (2013) Eur. J. Pharm. Sci., 48:259-71; Burnett et al. (2012) Chem Biol., 19:60-71; Magalhães et al. (2012) Mol. Ther., 20:616-24; Shigdar et al. (2011) Cancer Sci., 102:991-8; Thiel et al. (2012) Nucleic Acids Res., 40:6319-37).

In a particular embodiment, the anti-IDO2 aptamer is conjugated (e.g., directly or via a linker) to a compound (e.g., antibodies, peptides, proteins, nucleic acid molecules, small molecules, etc.) which targets the aptamer to a desired cell type and/or promotes cellular uptake of the aptamer (e.g., a cell penetrating moiety). The targeting moiety may be operably linked to the 5′ end, the 3′ end, or both ends or to internal nucleotides. In a particular embodiment, the targeting moiety and/or cell penetrating moiety are conjugated to the 5′ end and/or 3′ end. In a particular embodiment, the aptamer is conjugated to both a targeting moiety and a cell penetrating moiety. The aptamer may be targeted to a surface compound or protein (e.g., receptor) of a desired cell type (e.g., the surface compound or protein may be preferentially or exclusively expressed on the surface of the cell type to be targeted). As used herein, the term “cell penetrating agent” or “cell penetrating moiety” refers to compounds or functional groups which mediate transfer of a compound from an extracellular space to within a cell. For example, the cell penetrating moiety may be a cell penetrating aptamer (e.g., C1 or Otter (see, e.g., Burke, D. H. (2012) Mol. Ther., 20: 251-253)), cell penetrating peptide (e.g., Tat peptides, Penetratin, short amphipathic peptides (e.g., from the Pep- and MPG-families), oligoarginine, oligolysine), or a non-polar fluorescent group (e.g., a cyanine such as Cy3 or Cy5).

In a particular embodiment, the anti-IDO2 aptamer may be contained within a delivery vehicle such as a micelle, liposome, nanoparticle, or polymeric composition.

In a particular embodiment, the aptamer is complexed with (e.g., contained within or encapsulated by) a dendrimer (e.g., a cationic dendrimers such as poly(amido amine) (PAMAM) dendrimers or polypropyleneimine (PPI) dendrimer).

### Definitions

The following definitions are provided to facilitate an understanding of the present invention:

The singular forms “a,” “an,” and “the” include plural referents unless the context clearly dictates otherwise.

A “therapeutically effective amount” of a compound or a pharmaceutical composition refers to an amount effective to prevent, inhibit, treat, or lessen the symptoms of a particular disorder or disease. The treatment of an ocular disorder herein may refer to curing, relieving, and/or preventing the ocular disorder, the symptom of it, or the predisposition towards it.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the terms “host,” “subject,” and “patient” refer to any animal, including mammals such as humans.

The terms “immunosuppressant” and “immunosuppressive agent”, as used herein, include compounds or compositions which suppress immune responses or the symptoms associated therewith. Immunosuppressant include, without limitation, purine analogs (e.g., azathioprine), methotrexate, cyclosporine (e.g., cyclosporin A), cyclophosphamide, leflunomide, mycophenolate (mycophenolate mofetil), steroids (e.g., glucocorticoid, corticosteroid), methylprednisone, prednisone, non-steroidal anti-inflammatory drug (NSAID), chloroquine, hydroxycloroquine, chlorambucil, CD20 antagonist (e.g., rituximab, ocrelizumab, veltuzumab or ofatumumab), abatacept, a TNF antagonist (e.g., infliximab, adalimumab, etanercept), macrolides (e.g., pimecrolimus, tacrolimus (FK506), and sirolimus), dehydroepiandrosterone, lenalidomide, a CD40 antagonist (e.g., anti-CD4OL antibodies), abetimus sodium, BLys antagonists (e.g., anti-BLyS (e.g., belimumab), dactinomycin, bucillamine, penicillamine, leflunomide, mercaptopurine, pyrimidine analogs (e.g., cytosine arabinoside), mizoribine, alkylating agents (e.g., nitrogen mustard, phenylalanine mustard, buslfan, and cyclophosphamide), folic acid antagonsists (e.g., aminopterin and methotrexate), antibiotics (e.g., rapamycin, actinomycin D, mitomycin C, puramycin, and chloramphenicol), human IgG, antilymphocyte globulin (ALG), antibodies (e.g., anti-CD3 (OKT3), anti-CD4 (OKT4), anti-CD5, anti-CD7, anti-IL-2 receptor (e.g., daclizumab and basiliximab), anti-alpha/beta TCR, anti-ICAM-1, muromonab-CD3, anti-IL-12, alemtuzumab and antibodies to immunotoxins), and derivatives and analogs thereof.

As used herein, an “anti-inflammatory agent” refers to compounds for the treatment of an inflammatory disease or the symptoms associated therewith. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

A “cell-penetrating peptide” refers to a peptide which can transduce another peptide, protein, or nucleic acid into a cell in vitro and/or in vivo—i.e., it facilitates the cellular uptake of molecules. Examples of cell penetrating peptides include, without limitation, Tat peptides, penetratin, transportan, and the like.

The following example is provided to illustrate various embodiments of the present invention. The example is not intended to limit the invention in any way.

### EXAMPLE

Rheumatoid arthritis (RA), a debilitating condition characterized by inflammation of the synovial joints and eventual degradation of cartilage and bone, is an autoimmune disease. Although increased knowledge has favorably improved options for therapeutic management, like other autoimmune diseases, RA remains in need of treatments that can target disease more specifically (Buch et al., Ann. Rheum. Dis. (2011) 70:909-920; Helmick et al., Arthritis Rheum. (2008) 58:15-25; Upchurch et al., Rheumatology (Oxford) 51(Suppl 6): vi28-vi36). Long-standing evidence for reduced tryptophan levels and increased tryptophan catabolites in the serum and urine of patients with autoimmune disorders has implicated the tryptophan-catabolizing enzyme IDO in autoimmunity (Beetham et al., Proc. Soc. Exp. Biol. Med. (1964) 117:756-759; Labadarios et al., Rheumatol. Rehabil. (1978) 17:227-232; Mandel et al., Arch. Dermatol. (1996) 94:358-360; Spiera et al., J. Clin. Invest. (1969) 48: 856-859; Widner et al., Adv. Exp. Med. Biol. (1999) 467:571-577; Widner et al., Immunobiol., (2000) 201:621-630). Indeed, dysregulation of IDO has been directly correlated with disease activity in the autoimmune disorders RA and systemic lupus erythematosus (Furuzawa-Carballeda et al., Eur. J. Clin. Invest. (2011) 41:1037-1046; Pertovaara et al., Clin. Exp. Immunol. (2007) 150:274-278).

IDO has been known to have immunomodulatory effects since the unexpected discovery that IDO was necessary for maternal tolerance to fetal tissue (Munn et al., Science (1998) 281:1191-1193). Since then, it has been linked to immune modulation in a variety of diseases (Elovainio et al., Psychosom. Med. (2012) 74:675-681; Oxenkrug, G. F., Isr. J. Psychiatry Relat. Sci. (2010) 47:56-63; Xiao et al., Am. J. Respir. Crit. Care Med. (2013) 188:482-491), although its function is best established as a critical mediator of tumor immune evasion (Muller et al., Nat. Rev. Cancer (2006) 6:613-625; Prendergast et al., Curr. Med. Chem. (2011) 18:2257-2262). In these contexts, IDO is considered immunosuppressive. In the context of autoimmunity, however, the function of IDO is less clear. Several studies have demonstrated that IDO has an immunosuppressive role in inducible models of autoimmunity, such as trinitrobenzene sulfonic acid-induced colitis, collagen-induced arthritis, and experimental autoimmune encephalomyelitis (Gurtner et al., Gastroenterology (2003) 125:1762-1773; Sakurai et al., J. Neuroimmunol. (2002) 129:186-196; Szanto et al., Arthritis Res. Ther. (2007) 9:R50). Other models, including the KRN transgenic (Tg) [KRN (C57BL/6 x NOD)Fᵢ (K/BxN) and KRN.g7] mouse model of RA (Scott et al., J. Immunol. (2009) 182:7509-7517) as well as models of inflammatory airway disease (Xu et al., Proc. Natl. Acad. Sci. (2008) 105: 6690-6695), allergy (vonBubnoff et al., Allergy (2012) 67:718-725), and contact hypersensitivity (Metz et al., Int. Immunol. (2014) 26:357-67), have provided evidence that IDO plays a positive role in inflammatory responses. These models may be more relevant to inflammatory autoimmune disease in humans, given correlations of elevated tryptophan degradation with disease activity in autoimmune patients (Furuzawa-Carballeda et al., Eur. J. Clin. Invest. (2011) 41:1037-1046; Pertovaara et al., Clin. Exp. Immunol. (2007) 150:274-278). The contrasting results seen in the different models of autoimmunity and inflammation may reflect mechanistic differences in the disease induction process.

The KRN model is a spontaneous murine model of inflammatory autoimmune disease characterized by a rapid symmetrical onset of joint inflammation induced by the production of autoantibodies (Kouskoff et al., Cell (1996) 87:811-822; Korganow et al., Immunity (1999) 10:451-461). This model uses a TCR transgene, KRN, that when present in a genetic background expressing the I-Ag' MHC class II molecule, leads to the development of joint-specific autoimmune disease. In this model, the autoreactive T and B cells both recognize the glycolytic enzyme glucose-6-phosphate isomerase (GPI) as an autoantigen, and disease severity correlates with rising titers of anti-GPI Ig in the serum (Korganow et al., Immunity (1999) 10:451-461; Matsumoto et al., Science (1999) 286:1732-1735; Mandik-Nayak et al., Proc. Natl. Acad. Sci. (2002) 99:14368-14373). The K/BxN model has many features in common with human RA, including pathological changes in the joints, cellular infiltrates, proinflammatory cytokines, and autoantibody production (Kouskoff et al., Cell (1996) 87:811-822; Korganow et al., Immunity (1999) 10:451-461). However, as with all animal models, some differences can be noted. In particular, the specificity of the autoantibodies produced in K/BxN mice is to GPI rather than to rheumatoid factor or citrullinated proteins, the autoantibodies present in the majority of human RA patients (Mewar et al., Biomed. Pharmacother. (2006) 60:6480-655). As in human RA, arthritis in KRN mice is correlated with increased tryptophan catabolism, implicating the IDO pathway in the disease process (Scott et al., J. Immunol. (2009) 182:7509-7517).

Most previous studies of IDO and autoimmunity, including work demonstrating a reduced autoantibody response and an attenuated course of arthritis in the KRN-Tg mouse model of RA (Scott et al., J. Immunol. (2009) 182:7509-7517; Pigott et al., Arthritis Rheum. (2012) 64:2169-2178), have used the compound D/L-1-methyl-tryptophan (1MT) to inhibit IDO. Although widely considered an IDO inhibitor, 1MT, particularly the D-1MT stereoisomer, likely inhibits the IDO pathway rather than directly inhibiting the enzyme itself (Metz et al., Oncolmmunology (2012) 1:1460-1468). The IDO pathway is complex, and the mechanistic underpinnings of immune modulation are only beginning to be established (Prendergast et al., Curr.

Med. Chem. (2011) 18:2257-2262). Two closely related IDO genes, IDO1 and IDO2, appear to be inhibited by the different stereoisomers of 1MT (Metz et al., Cancer Res. (2007) 67:7082-7087) and may have different roles in immune regulation.

Conflicting evidence exists for the role of IDO in modulating the immune system. In cancer, IDO has generally been considered immunosuppressive, allowing for the expansion of regulatory T cell populations and suppression of T cell activation (Muller et al., Proc. Natl. Acad. Sci. (2008) 105:17073-17078; Yang et al., Transplantation (2007) 83:1643-1647), although recent work hints at a more complex immunomodulatory role (Prendergast et al., Cliff. Med. Chem. (2011) 18:2257-2262). The observation that IDO is upregulated in autoimmunity has been paradoxical, as increased immunosuppression would be predicted to be beneficial in this context, suggesting that the present understanding of the relationship between IDO activation and disease is incomplete. Unlike in cancer, in which IDO1 seems to be the major player in immune modulation (Smith et al., Cancer Discov. (2012) 2:722-735), other data directly implicate a second, related enzyme, IDO2, in immune system regulation in the context of autoimmunity (Merlo et al., J. Immunol. (2014) 192:2082-2090).

IDO2 is structurally related to IDO1, but its function is poorly established. Although IDO2 does catabolize tryptophan to kynurenine, it does so with substantially reduced efficiency compared with IDO1 (Metz et al., Cancer Res. (2007) 67:7082-7087; Yuasa et al., J. Mol. Evol. (2007) 65:705-714; Meininger et al., Biochim. Biophys. Acta (2011) 1814:1947-1954). IDO2 is expressed in a smaller range of tissues than is IDOL generally confined to liver, kidney, and epididymis, as well as APCs (e.g., dendritic cells) in immune tissues (Metz et al., Cancer Res. (2007) 67:7082-7087; Fukunaga et al., J. Histochem. Cytochem. (2012) 60:854-860). Even less clear is the mechanism by which IDO2 may influence the immune system. It is likely that the IDO pathway modulates the immune system indirectly, possibly through tryptophan depletion and sufficiency signals influencing GCN2 and mammalian target of rapamycin pathways (Metz et al, Oncolmmunology (2012) 1:1460-1468), although the relative contribution of IDO1 versus IDO2 to these signals is unknown.

Most previous studies evaluating the role of the IDO pathway in autoimmune responses used the small-molecule inhibitor D/L-1MT and have yielded conflicting results. Blocking IDO with 1MT exacerbates arthritis in collagen-induced arthritis (Szanto et al., Arthritis Res. Ther. (2007) 9:R50; Criado et al., Arthritis Rheum. (2009) 60:1342-1351) and experimental autoimmune encephalomyelitis (Sakurai et al., J. Neuroimmunol. (2002) 129:186-196), but alleviates disease in K/BxN and KRN.g7 arthritis as well as inflammatory airway disease models (Scott et al., J. Immunol. (2009) 182:7509-7517; Xu et al., Proc. Natl. Acad. Sci. (2008) 105:6690-6695). It is unclear why 1MT alleviates autoimmunity in some models yet exacerbates it in others. Because 1MT can inhibit both IDO1 and IDO2, one possible explanation is that it results from varying contributions of IDO1 and IDO2 in the different disease models. The specificity of the respective 1MT isomers for IDO1 versus IDO2 has also been controversial, with one report demonstrating that L-1MT inhibits IDO1 and D-1MT inhibits IDO2 (Metz et al., Cancer Res. (2007) 67:7082-7087), and another showing the direct opposite (Yuasa et al., Comp. Biochem. Physiol. B Biochem. Mol. Biol. (2010) 157:10-15). The L-isomer of 1MT appears to be a more direct enzymatic inhibitor of IDO in vitro; however, it is D-1MT, which affects IDO indirectly (Metz et al, OncoImmunology (2012) 1:1460-1468), that has a physiological effect on tumor progression in vivo (Hou et al., Cancer Res. (2007) 67:792-801).

IDO2 appears to work specifically to promote the development of autoantibodies but does not play an important role in directing Ab responses in general. Recently, it has been shown that IDO2 has a specific pathogenic function in the establishment and development of autoimmune arthritis in the KRN-Tg preclinical model of RA (Merlo et al., J. Immunol. (2014) 192:2082-2090). B cells from IDO2-deficient mice generate normal Ab responses to model Ags in vitro and in vivo. Thus, the mechanism for IDO2 may not be in the direct production of autoantibodies, but rather in providing T cell help to B cells to promote this autoantibody production. In support of this idea, IDO2 ko KRN.g7 mice have a general reduction in T cell help, with decreased Th1, Th2, and Th17 cell compartments and lower levels of the Th cytokines IL-4, IL-6, and IL-21. These cytokines have been shown to be important in driving B cell Ab responses, in particular by directing the differentiation and function of Tfh cells (Crotty, S., Annu. Rev. Immunol. (2011) 29:621-663). A trend toward lower levels of Tfh cells in IDO2 ko KRN.g7 mice was observed, although this did not reach statistical significance. IL-21, in addition to being associated with Tfh cells, is also produced by Th17 cells and is essential for the development of arthritis in the K/BxN model (Jang et al., J. Immunol. (2009) 182:4649-4656). The Th17 compartment is of particular interest in this situation, as IL-6, a regulator of Th17 cell differentiation, is reduced in IDO2 ko KRN.g7 mice, although IL-17 itself is not significantly altered. The role of Th17 cells in this model, however, has been difficult to elucidate. It has been shown that the presence of segmented filamentous bacteria in the gut of these mice drives Th17 production and is required for arthritis development (Wu et al., Immunity (2010) 32:815-827). However, adoptive transfer studies have yielded conflicting results on the contribution of Th17 cells to arthritis incidence. It has been demonstrated that adoptive transfer of Th17-polarized KRN T cells induces robust arthritis in recipient mice and that neutralization of IL-17A delays the onset of arthritis in this model (Hickman-Brecks et al., J. Autoimmun. (2011) 36:65-75). In contrast, it has also been found that arthritis development proceeds normally with T cells transferred from a mouse with inactivated expression of RORgt, the transcription factor that directs Th17 development (Block et al., J. Immunol. (2013) 191:2948-2955). A general reduction in differentiated T cell populations and cytokines in IDO2 ko KRN.g7 mice indicates that IDO2 mediates arthritis and autoantibody production by regulating the overall quality of T cell help, rather than by affecting a specific T cell subpopulation.

Given the reduction in T cell help, reciprocal adoptive transfer of T cells into T cell-deficient hosts was performed to directly test the effect of wt and IDO2 ko T cells. Herein, it is confirmed that it is the lack of IDO2 in the host mice that affects arthritis and autoantibody production, and not IDO2 in the T cells themselves. IDO2 may thus be acting in an APC in the host mice to influence both B and T cell activation. Although IDO1 is clearly important to dendritic cell function, especially in the ability of IDO-expressing dendritic cells to control the balance of effector and regulatory T cell populations required to maintain tolerogenic environments (Harden et al., Immunol. Invest. (2012) 41:738-764), the role of IDO2 in dendritic cells and other APCs such as B cells is largely unknown. Cross-talk between B cells and Th cells is necessary to generate effective T cell help for B cell Ab production. This cross-talk involves both cell surface molecules and soluble factors, including PD-1, ICOS, and IL-21 and their respective ligands (Nutt et al., Nat. Immunol. (2011) 12:472-477). It is possible that IDO2 is involved in directing one or more of these signals in autoreactive B cells. In support of this mechanism, CD4 T cells from IDO2 ko KRN.g7 mice express lower levels of IL-21. Another possibility is that IDO2 deletion in the recipient mice influences not just the activation, but also the survival, of the differentiated Th cell populations. For either scenario, in the absence of IDO2, T cell help and subsequent autoantibody production would be reduced, resulting in a diminished autoimmune response.

As explained herein, in experiments with IDO2-deficient mice, adoptive transplant experiments demonstrated that IDO2 expression in B cells was both necessary and sufficient to support robust arthritis development. IDO2 function in B cells was contingent on a cognate, Ag-specific interaction to exert its immunomodulatory effects on arthritis development. A similar requirement was confirmed in an established model of contact hypersensitivity, in which IDO2-expressing B cells are required for a robust inflammatory response. Mechanistic investigations showed that IDO2-deficient B cells lacked the ability to upregulate the costimulatory marker CD40, indicating IDO2 acts at the T-B cell interface to modulate the potency of T cell help needed to promote autoantibody production. Overall, the findings revealed that IDO2 expression by B cells modulates autoimmune responses by supporting the cross talk between autoreactive T and B cells.

In summary, direct evidence of a pathogenic role for IDO2 in driving B cell-mediated autoimmune disease is provided. Using the KRN preclinical model of RA, it is shown that IDO2 is required for the activation of CD4+Th cells, production of pathogenic autoantibodies, and subsequent development of arthritis. IDO2 appears to specifically regulate autoreactive responses, but not normal B cell responses, as IDO2 ko mice are able to mount productive Ab responses to model Ags in vitro and in vivo. Reciprocal adoptive transfer studies confirmed that autoantibody production and arthritis are mediated by IDO2 expression in a cell type extrinsic to the T cell, most likely an APC. Together, the data demonstrate that IDO2 contributes to autoimmunity via its role in autoantibody production, implicating IDO2 as an exciting new therapeutic target for RA.

### Materials and Methods

### Mice

KRN TCR Tg and IDO2-deficient (IDO2 ko) mice on a C57BL/6 background have been described (Kouskoff et al., Cell (1996) 87: 811-822; Metz et al., Int. Immunol. (2014) 26:357-67). Arthritic mice were generated by breeding KRN Tg C57BL/6 mice expressing the I-A^{g7} MHC class II molecule (KRN.g7). This process was repeated to generate arthritic mice lacking IDO2 (IDO2 ko KRN.g7). KRN.g7 mice develop arthritis with kinetics similar to that in the original K/BxN mice (Scott et al., J. Immunol. (2009) 182:7509-7517). All mice were bred and housed under specific pathogen-free conditions in the animal facility at the Lankenau Institute for Medical Research. Studies were performed in accordance with National Institutes of Health and Association for Assessment and Accreditation of Laboratory Animal Care guidelines with approval from the Lankenau Institute for Medical Research Institutional Animal Care and Use Committee.

### Arthritis Incidence

The two rear ankles of mice were measured starting at weaning (3 weeks of age). Measurement of ankle thickness was made above the footpad axially across the ankle joint, using a Fowler Metric Pocket Thickness Gauge. Ankle thickness was rounded off to the nearest 0.05mm.

### Enzyme-Linked Immunospot (ELISpot) Assay

Cells from the joint draining lymph nodes (dLNs) (axillary, brachial, and popliteal LNs) from 6-wk-old mice were plated at 4×10⁵ cells per well and diluted serially 1:4 in MultiScreen®-HA mixed cellulose ester membrane plates (Millipore) coated with GPI-his (10 mg/ml). The cells were incubated on the Ag-coated plates for 4 hours at 37° C. The Ig secreted by the plated cells was detected by alkaline phosphatase-conjugated goat anti-mouse total Ig secondary Ab (Southern Biotechnology Associates) and visualized using NBT/BCIP substrate (NBT/5-bromo-4-chloro-3-indolyl phosphate; Sigma-Aldrich).

### Results

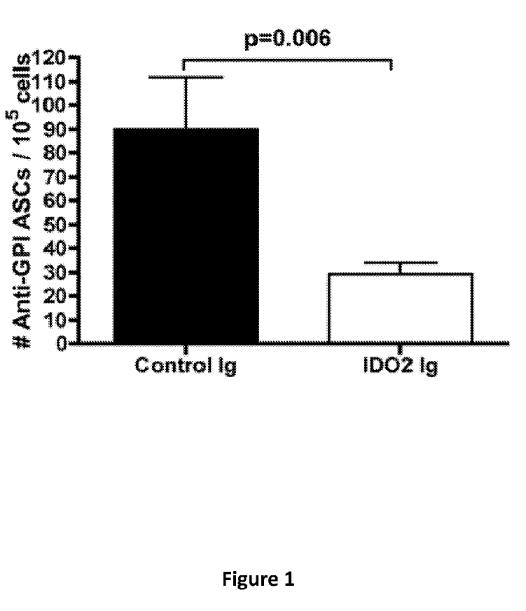

K/BxN mice were injected i.p. with 0.5mg control mouse Ig or anti-IDO2 Ig at 21 days of age. At 6 weeks of age, the joint draining lymph nodes were harvested and analyzed for the number of autoantibody secreting cells (ASCs) by enzyme-linked immunospot (ELISpot) assay. FIG. 1 shows that anti-IDO2 antibodies inhibit autoantibody production.

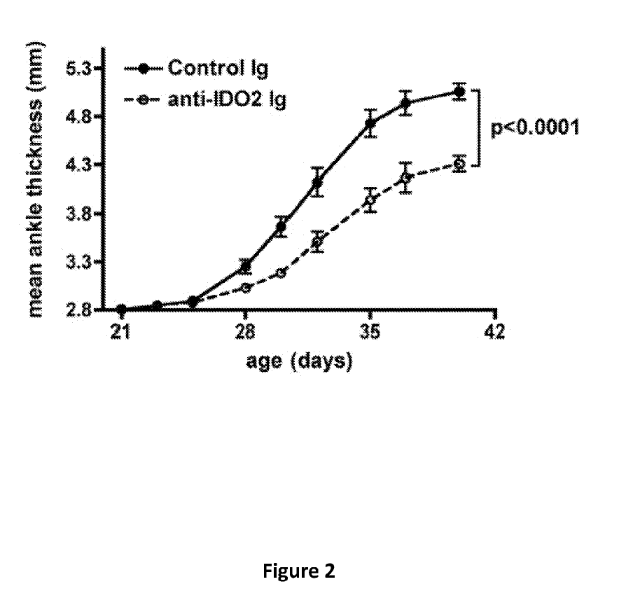

To assay the ability of anti-IDO2 antibodies to inhibit arthritis, K/BxN mice were injected with 0.5mg control mouse Ig or anti-IDO2 Ig at 21 days of age. The mice followed for the development of arthritis by measuring ankle thickness. As seen in FIG. 2, anti-IDO2 antibodies inhibit joint inflammation and delay the onset and attenuate the severity of arthritis.

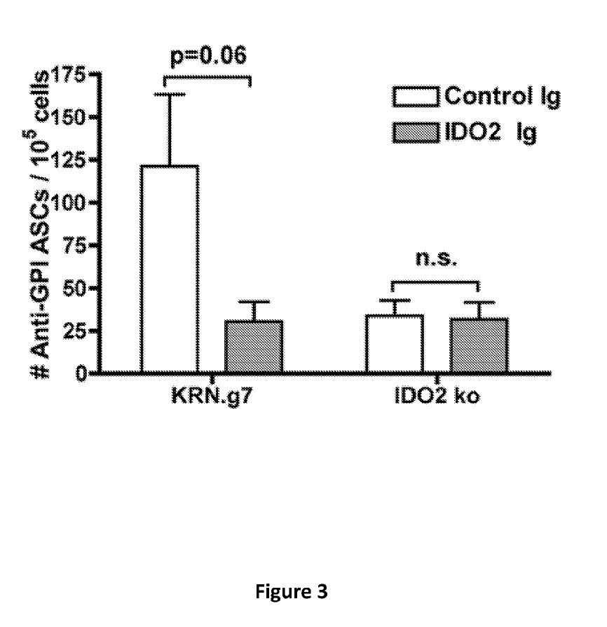

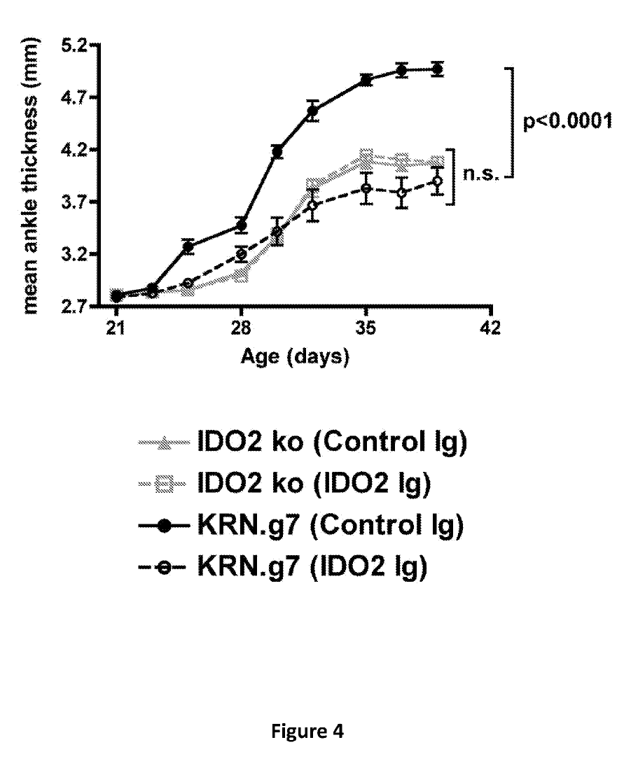

The specificity of the anti-IDO2 antibody was confirmed by the lack of a response in mice genetically deficient for IDO2. Briefly, KRN.g7 or IDO2 knockout (ko) KRN.g7 mice were injected with 0.5mg control mouse Ig or anti-IDO2 Ig at 21 days of age. At 6 weeks of age, the joint draining lymph nodes were harvested and analyzed for the number of autoantibody secreting cells (ASCs) by ELISpot assay. As seen in FIG. 3, the administration of anti-IDO2 antibodies failed to yield a change in autoantibody levels in IDO2 knockout mice. Further, KRN.g7 or IDO2 ko KRN.g7 mice were injected with 0.5mg control mouse Ig or anti-IDO2 Ig at 21 days of age and followed for the development of arthritis. As seen in FIG. 4, the administration of anti-IDO2 antibodies failed to yield a change in inflammation or the progression of the arthritis in IDO2 knockout mice.

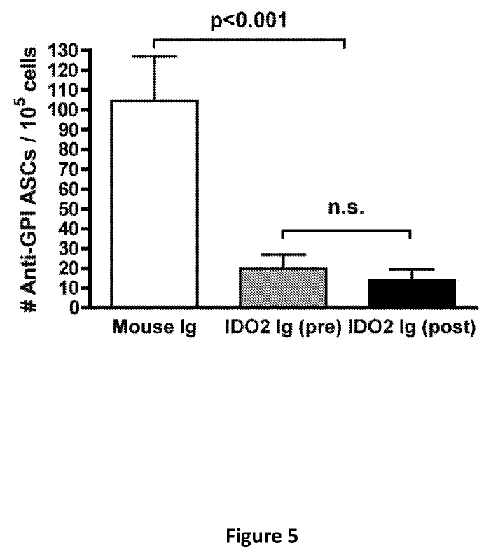

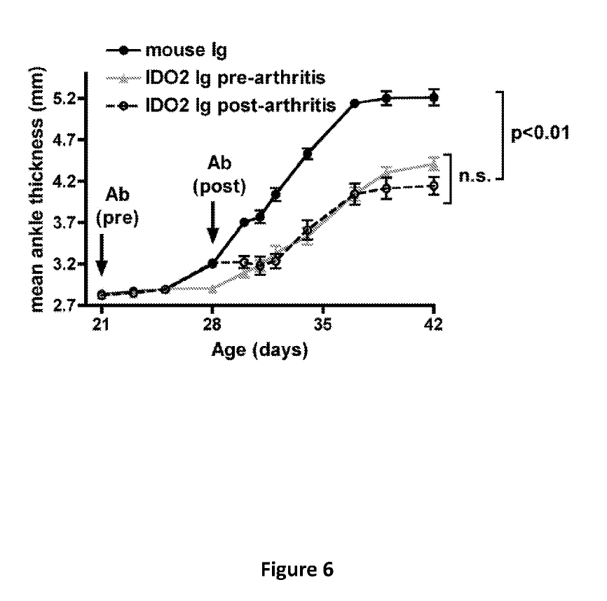

The ability of the anti-IDO2 antibodies to inhibit autoantibody production when administered after the onset of arthritis was also shown. As seen in FIG. 5, anti-IDO2 antibodies inhibit autoantibody production when administered before or after arthritis onset. Further, the administration of anti-IDO2 antibodies inhibits joint inflammation when administered before or after the onset of arthritis (FIG. 6).

Several publications and patent documents are cited in the foregoing specification in order to more fully describe the state of the art to which this invention pertains. The disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating, inhibiting, and/or preventing an autoimmune disease or disorder in a subject in need thereof, said method comprising administering at least one anti-IDO2 aptamer and/or anti-IDO2 antibody or fragment thereof to said subject.

2. The method of claim 1, wherein said method comprises administering at least one anti-IDO2 antibody or fragment thereof to said subject.

3. The method of claim 1, wherein said autoimmune disease or disorder is a cancer which is sustained by antibody secretion.

4. The method of claim 1, wherein said autoimmune disease or disorder is an antibody-mediated paraneoplastic syndrome.

5. The method of claim 1, wherein said autoimmune disease or disorder is an antibody-mediated inflammatory disease.

6. The method of claim 1, comprising administering a composition comprising at least one anti-IDO2 aptamer and/or anti-IDO2 antibody or fragment thereof and at least one pharmaceutically acceptable carrier.

7. The method of claim 1, wherein said anti-IDO2 aptamer and/or anti-IDO2 antibody is immunologically specific for SEQ ID NO: 1.

8. The method of claim 1, wherein said anti-IDO2 aptamer and/or anti-IDO2 antibody is immunologically specific for amino acids 331-351 of human IDO2.

9. The method of claim 1, wherein said anti-IDO2 aptamer and/or anti-IDO2 antibody is immunologically specific for SEQ ID NO: 2.

10. The method of claim 1, wherein said anti-IDO2 aptamer and/or anti-IDO2 antibody is immunologically specific for SEQ ID NO: 5.

11. The method of claim 1, wherein said method further comprises the administration of at least one anti-inflammatory.

12. The method of claim 1, wherein said autoimmune disease is rheumatoid arthritis.

13. The method of claim 3, wherein said method further comprises the administration of at least one chemotherapeutic agent.

14. A method for reducing autoantibody production in a subject, said method comprising administering at least one anti-IDO2 aptamer and/or anti-IDO2 antibody or fragment thereof to said subject.

15. The method of claim 14, wherein said anti-IDO2 aptamer and/or anti-IDO2 antibody is immunologically specific for SEQ ID NO: 1.

16. The method of claim 14, wherein said anti-IDO2 aptamer and/or anti-IDO2 antibody is immunologically specific for amino acids 331-351 of human IDO2.

17. The method of claim 14, wherein said anti-IDO2 aptamer and/or anti-IDO2 antibody is immunologically specific for SEQ ID NO: 2.

18. The method of claim 14, wherein said anti-IDO2 aptamer and/or anti-IDO2 antibody is immunologically specific for SEQ ID NO: 5.


SEQUENCE LISTING (5 sequences)

SEQ ID NO: 1 - protein, 420 aa, Homo sapiens
MLHFHYYDTSNKIMEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

SEQ ID NO: 2 - protein, 21 aa, Artificial Sequence
  IDO2 epitope
RDYILASGPGDCLMAYNQCVE

SEQ ID NO: 3 - protein, 128 aa, Artificial Sequence
  anti-IDO2 antibody light chain
MSVLTQVLALLLLWLTGARCDIQMTQSPASLSASVGETVTITCRASENIHNYLAWYQQKQGKSPQLLVYNPKNLADGVPSRFSGSGSGTQYSLNINSLQPEDFGTYYCQHFWNTPPTFGGGTRLEIKR

SEQ ID NO: 4 - protein, 153 aa, Artificial Sequence
  anti-IDO2 antibody heavy chain
MSSPQTLNTLTLTMGWSWIFLFLLSGTAGVLSEVQLQQSGPELVKPGASVQISCKTSGYTFTEYTMHWVKQSHGKSLEWLGIIHPDNGITRYNQKFKAKATLTEDKSSRTAYMELRSLTSEDSAVYYCARRYYGNFDYALDYWGQGTSVTVSS

SEQ ID NO: 5 - protein, 21 aa, Artificial Sequence
  IDO2 epitope
RDYILSSGQDHLLTAYNQCVQ